(19) United States
(12) Patent Application Publication
(10) Pub. No.: US 2022/0124960 A1
(43) Pub. Date: Apr. 28, 2022

(54) Commercial Farm Optimization Utilizing Simulation, Remote Sensing, and Farmer Inputs

(71) Applicant: James Canyon, San Diego, CA (US)

(72) Inventor: James Canyon, San Diego, CA (US)

(21) Appl. No.: 17/079,374

(22) Filed: Oct. 23, 2020

(51) Int. Cl. A01B 79/00; G01N 27/22; G01S 13/88; G01S 13/95; G01S 13/90; G01S 19/39

(52) U.S. Cl. CPC A01B 79/005; G01N 27/223; G01S 19/39; G01S 13/95; G01S 13/90; G01S 13/885

(57) ABSTRACT

Briefly, an advanced data collection and processing system is provided to collect multiple types of data from farm terrain to drive a farm management processes, including a crop yield simulation tool. The system has disparate sensors mounted on a vehicle, such as a ground vehicle or airplane, which collects data from GPS, RADAR, camera, thermal, LiDAR and spectral scanners for an area of interest on a farm terrain. The system also collects data from public sources and the farm manager, which enable the simulation tool to accurately predict crop growth and maturity.

### RELATED APPLICATIONS

This application claims priority to U.S. provisional patent application No. 62/924,845, filed Oct. 23, 2019 and entitled “Commercial Farm Optimization Utilizing Simulation, Remote sensing, and Famer Inputs.” This application is also a continuation in part to U.S. patent application Ser. No. 16/983,204, filed Aug. 3, 2020, which is a continuation of U.S. patent application Ser. No. 16/035,612, filed Jul. 14, 2018, now U.S. Pat. No. 10,729,059, which is a continuation of U.S. patent application Ser. No. 15/057,885, filed Mar. 1, 2016, which claims priority to U.S. provisional patent application 62/127,243. This application is also a continuation in part to U.S. patent application Ser. No. 16/253,152, filed Jan. 21, 2019. This application is also a continuation in part to U.S. patent application Ser. No. 16/747,795, filed Jan. 21, 2020. All of which are incorporated herein by reference, as if set forth in their entirety.

### FIELD OF THE INVENTION

The field of the invention is data collection systems to support commercial farm simulation software tools.

### BACKGROUND

Farming is a difficult business, margins are low and successful fanners continuously look for methods to optimization production yield while keeping production costs low. The advancements described herein highlight three new important aspects of advanced farm production: (1) farm process, (2) soil optimization, and (3) a novel farm crop yield simulator. Certain fundamental advancements regarding remote sensing of farm data are set forth in other co-pending applications: U.S. patent application Ser. Nos. 16/983,204; 16/253,152; and 16/747,795, all of which are incorporated herein by reference, as if set forth in their entirety. This application focuses on the system required to acquire farm data, deliver the data to the cloud, process the data and deliver a crop yield simulation tool to fanners and agronomists.

Most of the world is suffering in a chronic state lacking fresh drinking water. This leads to a shortage of water for agriculture, which makes it expensive or impossible to grow crops effectively. Increased need for water conservation in recent years has led to higher food prices and higher costs for farmers and consumers alike. The need for conservation has stemmed from higher demands on food production and higher population bases in localized areas. Water authorities around the United States, and the world are enacting watering limits and water usage expectations to ensure the valuable resource is being used carefully. In addition to agricultural needs, residential, sporting and landscaping all consume water at an alarming rate. It has been shown that in commercial crops, the amount of water used will greatly affect the profitability of the farm and therefore fanners are economically motivated to use the water carefully.

It would be desirable therefore to have an innovative sensor technology such that an accurate watering and fertilizing regime can be constructed to optimize water use and minimize over fertilization runoff. Large areas can be monitored and optimized at extremely low costs utilizing proposed remote sensing technologies described herein, thereby improving the production of food and other agricultural products. Since it is clear that water conservation is important for society, this invention describes a method and apparatus to be able to enable optimal water and fertilizer usage for a given landscape or crop. The subject of this invention is to, for a given crop or landscape, enable the water user to reduce the water usage to the optimal point and therefore minimize the cost of water, and/or optimize the yield in the growing of commercial food crops.

In order to enable this ability several pieces of technology are necessary. Some of the technology has been developed and some of the technology is the subject of this invention. In order to optimize cost further, technology choices were made to enable the optimal cost structure. Other choices could yield similar results in terms of water usage and therefore could still result in significant savings for the user, however they would not yield the ideal cost savings.

The advent of a crop yield simulator made inexpensive and requiring no on farm equipment and not requiring a team of agronomists to gather information in a university laboratory setting is un-known, as universities around the world use similar systems requiring sensors on farm, and a team of agronomists scour the farm to gather data required to operate the simulator.

Companies utilizing other systems utilizing aircraft, or satellites to predict yield, unfortunately have fallen short due to variation caused by the individual farm soil parameters, which require expensive 3rd party sensors and/or even more expensive agronomists performing periodic visits to gather missing information otherwise defined by this paper.

### SUMMARY OF THE INVENTION

The present invention describes a system, apparatus, and method designed to reduce production costs and improve production yields for commercial farmers through the use of a crop yield whole farm simulation tool, the heart of the tool being crop yield simulator described herein. The system described utilizes remote sensing equipment mounted onto vehicles in air and/or on ground to measure most all parameters required to run today's modern farm. We believe aircraft is most efficient method of measuring due to the fact aircraft fly fast (compared to ground, drone, satellite methods), and are in near proximity to the farm thus provide high resolutions (compared to satellite methods), and allow the use of relatively inexpensive sensing equipment, and permit high scan rates (>100,000 acres per day), and keep scan costs low, and or are synergistic with the use of existing aircraft such as crop dusters, and small aircraft, and allow for very high farm measurement resolutions. The system described herein is also applicable to scanning with other air and or ground based vehicles such as satellites, and or drones, and or tractors, to name a few specific devices.

The system described here deploys various sensors and devices used in conjunction to determine key aspects of agriculture production including farm practices, and land management, and crop farm management. The crop farm management piece is highlighted by our crop yield simulation tool which utilizes inputs derived by the sensor set described herein. Inputs from the sensor suite described herein are used to develop higher order inputs used by the simulator. Examples of results derived by multiple inputs include farm microclimate, farm crop health, farm crop stress, and soil properties including soil type and soil moisture but are not limited to these quantities.

Farm terrain is mapped three dimensionally from the tip of the crop to the depth of the ground penetrating radar utilizing a group of sensors described herein. Mapping resolutions can be viewed as an assembly of individual two- or three-dimensional pixels assembled to represent the farm under measurement. The measured volume consists of a group of individual sensor measurements whereby each individual pixel in three-dimensional space is cataloged by location and consists of measurements from multiple sensors described by the sensor group.

The sensor group required to deliver a minimum working system include a compliment of individual sensors which have different focal lengths and spot sizes and are subject to different mechanical requirements. Key to the implementation of this system is system ability to manage the different focal lengths, spot sizes and mechanical requirements required to image a single farm location as described by a volume or surface in three-dimensional space.

The three-dimensional space of each pixel is defined by the system and can be described by the ground surface contour of the spot at location of pixel in x,y moving from max height to max depth. The max height is defined by the microclimate above the crop. The max depth is defined by the penetration depth of the radar in soil (typically defined by the depth of the saturated region, or 8′ deep, or 30-48″ in agriculture soils in the US).

The minimum sensor set consists of thermal imaging, Spectroscopy, Visual images, GPS, and radar defined by the referenced patent(s). The addition of LiDAR allows more accurate irrigation and rain runoff models.

The system described herein is capable of delivering a vast amount of information that catalogs and delivers the best university-proven practices to optimize crop production, and crop health and crop yields and delivers this data to farm managers equally operating large and small farms inexpensively and requiring a minimum of farm manager input.

Because the tool described herein scans each farm and provides advice and measurements specific to each farm in its database, and delivers a simulation and planning tool which allow farm managers the ability to simulate yields and plan their crop growing a season in advance and take into account farming practices, and soil preparation, and seed type, and planning for nutrient application, and planning for pest applications, and be able to adjust these parameters as a function of simulated crop yield suggests this tool is powerful.

In addition to optimizing the production of crops using data gathered by remote sensing defined by this patent, such a tool is capable of suggesting lower cost production methodologies, yield comparisons between different seed manufacturers, optimization of irrigation methodologies, and optimization of nutrient methodologies to name a few.

When the system defined here is fully built out, it is expected the benefits delivered to the farm manager will greatly outweigh the cost of use projected to be roughly 10% the price of seed.

The advancement here consists of five major elements: (1) advanced remote sensing vehicle; (2) cloud storage and computer software resources to “develop” images in conjunction with other external data sets required by the farm optimization framework; (3) a farm optimization software framework; (4) models used by the farm optimization framework; (5) and a farmer interface and feedback module. It will be understood that not all five advancements need to be included in every embodiment of the invention.

Definitions of vehicles capable of measuring microclimates, crop health, and soil properties consist of both ground-based vehicles as well as aircraft. Hardware required to make the measurements are sensitive and contain various focal points and volume resolutions and therefore must be managed differently to maintain high quality measurement capabilities. Some of the equipment is sensitive to vibrations therefore equipment location and dampening is another consideration discussed herein.

Measurement location within this system is very important and thus may in some embodiments require an order of magnitude better location stamp over GPS with a goal to achieve centimeter location accuracy in x,y and z. This optimization requires multiple sensor inputs to achieve a full complement of measurements required by the system

Synchronization and alignment of multiple disparate sensors is an important aspect to data collection. The compliment of remote sensing equipment consists of one or all of the listed sensors including LiDAR for mapping surface contours, cameras to record visual images, infra-red cameras to record thermal images, one or more spectrometers to record very accurate spectra typically in the 780-2500 nm range, Radar for measuring soil type and soil moisture, climate sensor which measures temperature and humidity. Measurements from this suite of sensors are calibrated to deliver measurements of one location, each of the measurements are then date/time and location stamped using a combination of GPS and or visual imagery.

Alignment of the disparate measurement systems to a single location in X,Y, and Z is required to ensure repeat measurement accuracy, to maintain focal points, and to provide clear imagery. This process is performed utilizing three methods: Alignment of disparate measurement systems, optimization of measurement location, and minimization of noise due to vehicle vibrations.

Alignment of the disparate measurement systems—is crucial and is performed in one or two steps depending on application. The first step is on vehicle. All sensing instruments are connected to a single controller. The controller is seeded with a preferred three-dimensional path. As the vehicle attempts to travel along the preferred path the controller measures the actual path using GPS. The controller then consults the preferred path, the pre-determined measurement location, the existing location, and the resolution of each instrument and latency then instructs each instrument when to make a measurement. As measurements are recorded the controller time/date/3D location stamps each measurement and stores the data either in its memory or in the sensor memory for future upload to the cloud.

The vehicle travels a pre-determined path. Due to the nature of the subject matter, the various equipment resolutions and focal point requirements, and time varying location of measurement vehicle (vehicle is typically never in the proper place to make a consistent measurement over time), measurement complexities arise. The most detailed measurement is LiDAR (the highest frequency) and the least detailed measurement is RADAR (the lowest frequency). Focal points vary as a function of position, scan rates vary and requires a smart controller to manage the mayhem to ensure each measurement is made at the right time and focal point.

Minimization of Vibration will often be required to improve quality for optical and other measurements. As measurement frequency move from the 100s of MHz range to 100s of THz range, measurement accuracy (blurred measurement) are strongly affected by vibrations. In addition to synchronizing timing and focal points of our system, sensors must be mounted on vibration stabilized tables designed to attenuate vibrations in X,Y, and Z thus improving measurement accuracy in all three axis.

Optimization of measurement location utilizing visual imagery may be used to increase data collection resolution. In the system described, it is imperative location accuracy is maintained. Employing the use of GPS and visual imagery to improve location accuracy is discussed and can be implemented in real time or post processing two a visual images, with a reference and the current location visual image for each GPS location thus generating an error vector. In real time, the error vector is fed into the location computer and an offset is derived prior to measurements. In post processing the error vector is derived, and all measurements are shifted by the offset, and although not as accurate is a viable and will probably be implemented into the system first due to cost of R&D. The process of improving GPS accuracy from meters to centimeters utilizes a cataloged reference photo, GPS location, and a current measurement photo. An error vector is derived by comparing the reference photo of each GPS measurement location to a current photo of the area. Offset vector is derived for each measurement location by aligning the reference location photograph with the newly received photograph.

### BRIEF DESCRIPTION OF THE DRAWINGS

FIG. 100 is an illustration of farm practice, farm soil and farm crop yield simulation in accord with an embodiment of the present invention.

FIG. 200 is an illustration of a farm croup management process flow diagram in accord with an embodiment of the present invention.

FIG. 300 is a crop management information flow chart in accord with an embodiment of the present invention.

FIG. 400 is an illustration of a cloud simulator framework and data flow in accord with an embodiment of the present invention.

FIG. 500 is a diagram of the vehicle complement of equipment in accord with an embodiment of the present invention.

FIG. 600 is a table of remote sensing equipment relationships in accord with an embodiment of the present invention.

FIG. 700 is an illustration of a controller alignment of disparate sensors in accord with an embodiment of the present invention.

FIG. 800 is an illustration of an optical instrument stabilization and housing design in accord with an embodiment of the present invention.

FIG. 900 is an illustration of position vector error correction in accord with an embodiment of the present invention.

### DESCRIPTION

Governments have spent billions in an attempt to support increased crop yields, crop modelling, and a wide variety of agriculture related activities. This spending is typically developed in universities and private institutions who utilize it for a wide variety of things. Yet to date, wide adoption of crop yield optimization tools are not used by even the large corporate farms in a standalone fashion and are rarely used by small farmers.

Currently the largest such simulation tool is APSIM or Agricultural Production Systems sIMulator. APSIM Initiative is a collaboration between Australia's national science agency CSIRO, the Queensland Government, The University of Queensland, University of Southern Queensland, and internationally with AgResearch Ltd in New Zealand, and Iowa State University in the United States. From humble beginnings twenty years ago, APSIM is evolved to a framework containing many of the key models required for modelling crop growth on single and multi-field farms.

With 20 years of development and worlds most used model, in 2017 APSIM adoption was <487 users in Australia and <250 users in the United States. Unfortunately, most commercial farms do not possess enough data to run APSIM nor do they know what to do with the data once it is calculated. This demonstrates the large gap between good intentioned governments and the fanner.

The invention solves the problem by opening up a simple discussion with individual farm manager/agronomists first utilizing Farm Practice Optimization 110, then Farm Soil Optimization 120. These inputs are required to run the whole Farm Management Optimization module 130.

Farm Soil Optimization module is comprised of four sub-modules and is designed to open a discussion with the farm manager to inform and optimize soil management, and irrigation, and soil nutrient management on his/her farm. This module uses inputs from the first module, Farm Practice optimization 110 and focuses on soil type 122, surface flatness 124, soil drainage 126, and nutrient application 128. The software will deliver maps of soil type and soil moisture as a function of time from previous farm scans held in the cloud database. The purpose of this module is to inform and help the farm manager optimize the farming process.

Soil type 122 is the first sub module delivered in the farm soil optimization module. Images of the farm soil type in three dimensions are provided to give the farm manager detailed description of what is happening beneath the soil surface. Software will identify possible issues with clay or other impediments given prior inputs such as surface flatness, irrigation methodology etc.

Soil Drainage 126 is the second sub module delivered in the farm soil optimization module. The module delivers review water transport (movement vs time) in the farmland. Highlighted are insufficiently irrigated regions in soil plots with suggestions to add either adjust irrigation or add additional drainage. In areas where fanners are adding tiles (drainage pipes) to drain water away from the farmland, the simulator will suggest where the old system may not be working and suggest areas which require additional drainage.

Surface Flatness 128 is the third sub-module. This module delivers the farm surface in three dimensions and is very important for fanners who flood irrigate. This very accurate representation will suggest when areas of the farm are not level or have adequate slope.

Nutrient application 129 is the last sub-module and prepares the farm for the use of the crop yield simulation. Nutrient application and soil preparation prior to planting is very important as it drives both production yield and over application of fertilizer becomes unwanted farm runoff.

Optimization of crop yield 130 requires optimization of farm practices 110, and optimization of land use 120 prior to use. Information required of the fanner to complete these modules is easily delivered. Once completed, the whole farm simulator has enough information to operate thus delivering the ability to suggest optimum seeding dates based on seed type and manufacturer, or profit as a function of seed manufacturer to name two.

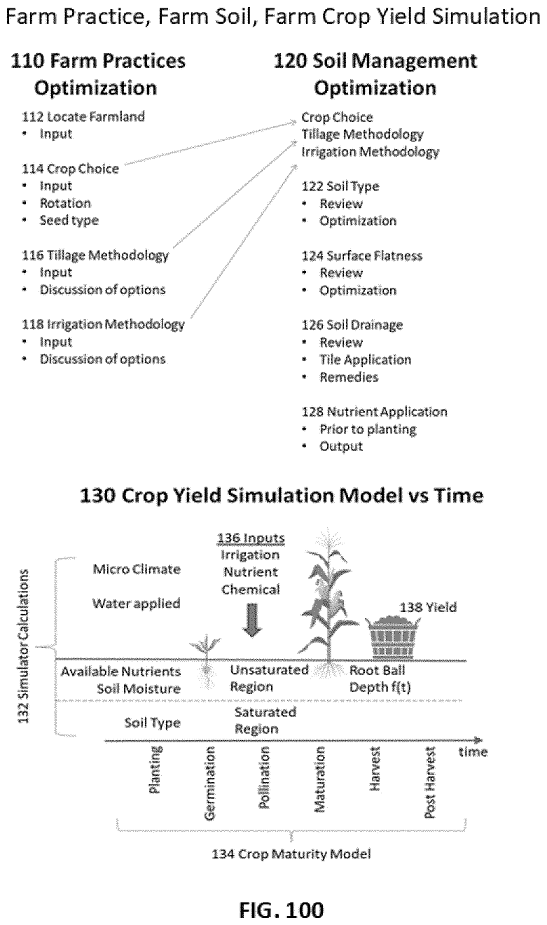

Crop Simulation 130 is the third and last module delivered by the system as shown in FIG. 100, which shows a schematic of crop growth inside soil from soil prep through post-harvest. Simulator calculations 132 shows a schematic diagram of measurements made by the sensors. Maturity model 134 shows crop maturity vs time as simulated in the simulator. Inputs 136 shows key inputs form separate sources including farm manager inputs. Lastly yield 138 shows resultant farm yield. The resultant simulator allows the farmer the luxury of planning his crop using a plant date and models of climate and soil type and soil moisture as a function of time to simulate the growth of his crop through harvest. Variables such as irrigation times and amounts, irrigation methodology, nutrient application plan and rate, chemical application plan are all loaded into the simulator and adjust yield output thus allowing the farm manager the ability to optimize his yield and profit.

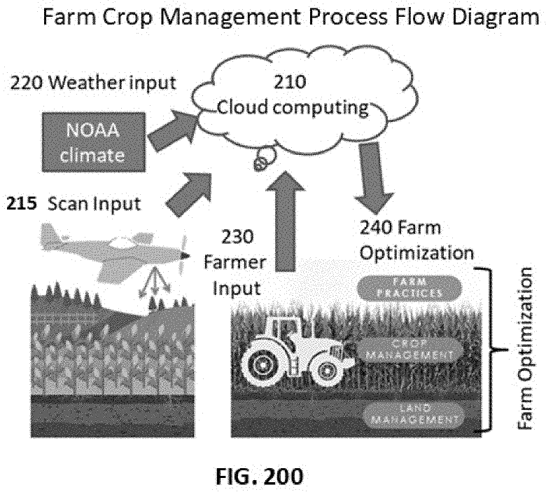

Referring now to FIG. 200, a farm crop management process flow diagram is shown. Cloud 210 is a representation of the cloud or a simplified version of a computer with storage and access to multiple data sets as defined later in this document. Inputs 220 to the cloud computer 210 are updated weather information for the area under question. It will be appreciated that other inputs may be used. The sensor package in the scanner is deployed in an aircraft or ground vehicle and generates scan input 215 data for the farm terrain, and when it has completed its measurements data is uploaded into the cloud 210 for processing. Farmer input 239 is how the fanner informs the tool about his farm and farming processes. Once information is uploaded into the cloud 210 or computer, the computer can prepare advise for farm optimization 230. Farm optimization 230 is broken down into three areas, farm practice, land management, and crop management as depicted in the figure.

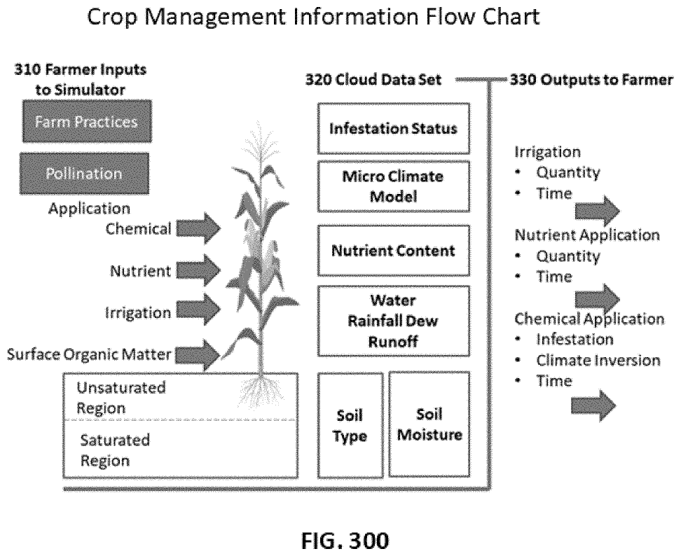

Referring now to FIG. 300, a crop management information flow chart is illustrated. Farm inputs 310 summarizes fanner inputs to simulator. These inputs include farm practices, pollination methodology, farm application of: irrigation, nutrients such as fertilizer, and chemical such as pesticides. These inputs also include surface organic matter if any and farm location.

Cloud data set 320 summarizes remote sensing cloud data set. This data set includes soil type, soil moisture, water applied due to rainfall, and dew, measured nutrient content, a model of microclimate and determination of infestation status.

Outputs to fanner 330 summarizes the output of the cloud computer. These outputs come in two types; the user can interrogate some measured data (not shown in picture) The second types of outputs are outputs delivered from the simulation tool. These outputs include suggested irrigation quantity and date, nutrient application quantity and date, and lastly chemical infestation status, climate inversion dates, and suggested times for application.

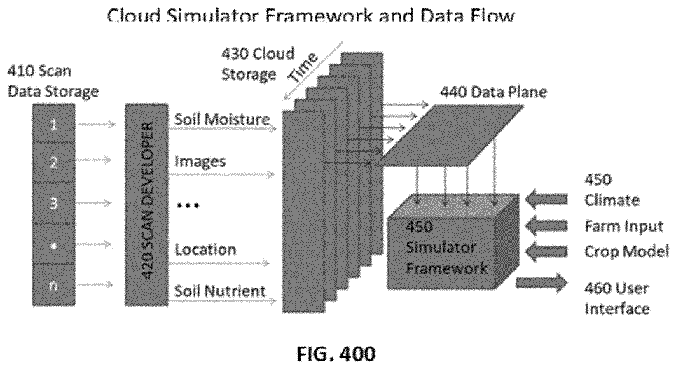

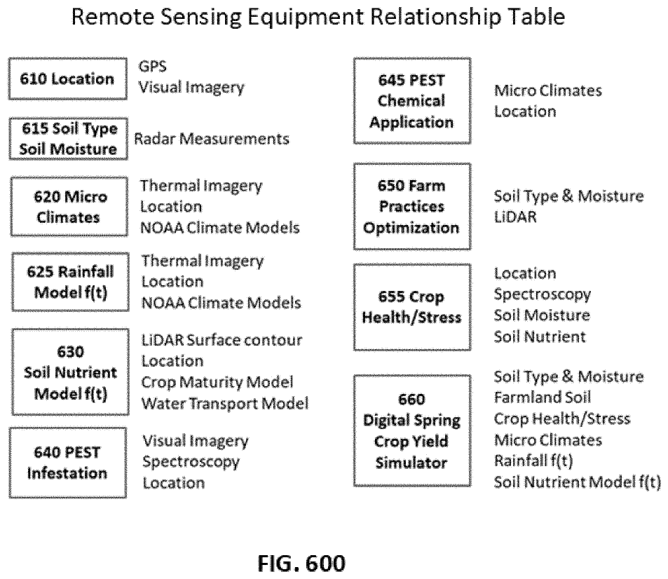

Referring now to FIG. 400, a data flow and cloud simulator process are diagramed. Individual raw scanned data is delivered to the cloud and stored in the data scan memory 410. Data is read in by the scan developer computer which selects individual scan data required to deliver a single output parameter using the algorithm defined in FIG. 600. A simple example of this is to look at the location output. In order to output location, the scan developer must interrogate GPS+Visual image for each location measurement. This output is then stored into cloud storage 430 measurement. Some outputs require a processed parameter plus multiple raw data inputs. An example of this is soil nutrient and the subset of this being nitrogen. Nitrogen measurement requires a location stamp (data 430) plus spectroscopic information (data 410).

After all measured data has been processed and stored it is available for the farm crop yield simulator 450. Data 450 is accessed from the cloud storage 430 plus uses inputs from other sources (climate, farm manager input). Farmer interface 460 directs which software module used by the simulator. Software modules are generated by multiple institutions.

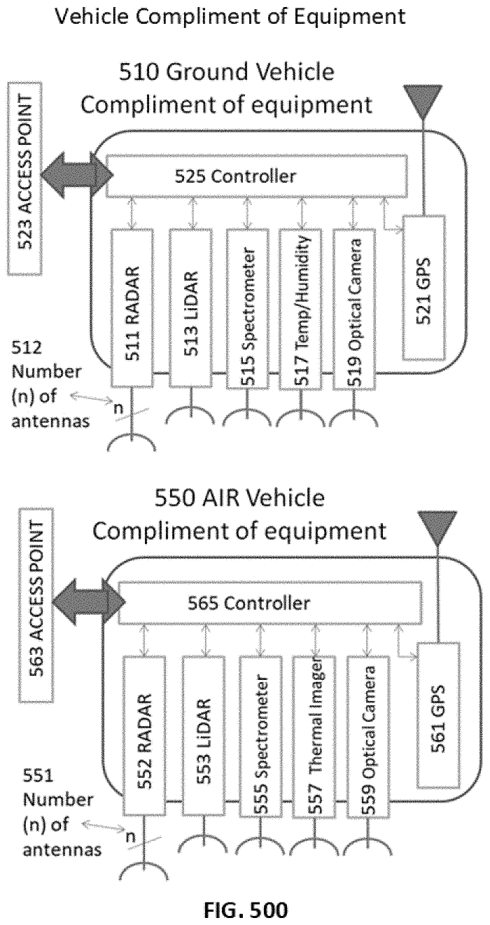

FIG. 500 shows the minimum compliment of sensing devices required to scan farmland required by in some embodiments of the system. Two vehicles are introduced: a ground vehicle and an air vehicle. Air vehicles include satellites, drones, helicopters, and fixed wing aircraft. Each equipment set is managed and run using a computer 525/565, which performs many tasks from location determination to scheduling, to uploading data to name a few. The computer all flight information is loaded to the computer prior to flight. Once loaded the computer is capable of executing the measurement plan autonomously. Once the flight is completed, data is delivered to the cloud via the access point 523/563 which can be a wireless technology, a wired technology, or a hand carried storage device.

GPS location is determined using GPS sensors 521/561 that connect to the controller 525/565. The compliment set of sensors required to perform farm sensing is also described in FIG. 500 and includes Radar 511/522, and one or multiple spectrometers 515/555 and optical cameras 519/559. Air vehicles may require an additional thermal imager 557. Ground vehicles may require an additional temperature and humidity sensor 517. LiDAR 513/555 may be added if local LiDAR data is not available.

The controller computer 525/565 is loaded with a route with locations for measurement. The controller 525/565 measures GPS and uses visual imagery to determine true location. Once the device is at the location required the controller signals measurements from different instruments as a function of their spot size. After each measurement, the controller retrieves the data and time/date/location stamps each measurement and stores it inside local memory. Once information from the access point is ready to upload data, the controller uploads its data and is ready for the next set of measurements.

FIG. 600 diagrams the sensors required to generate each parameter required by the simulator. Most sensor systems require multiple inputs from multiple sensors to develop an accurate result. This system is no different and as such we have defined a minimum set of sensors which will achieve our goals of modeling a modern farm. Our current system consists of approximately 6 sensor types which consist of Radar, LiDAR, Spectrometry, Thermal imager, Optical imager, and GPS. Typically, the spectrometer consists of multiple boxes which focus on separate frequencies required to scan the full band. FIG. 600 diagrams which sensors are required for each parameter. It is important to note that ALL sensors minus LiDAR (noting some states have extensive LiDAR maps which invalidate the need to add the sensor) are required to operate the crop yield simulation tool of FIG. 660. Each sensor is discussed below:

Location 610—Location requires GPS+Visual imagery as a minimum set of inputs.

Soil type 615—Soil Moisture and Soil Type requires radar imagery as a function of time as defined in the reference patents.

Microclimates 620—Microclimate Models require input from Location, thermal imagery and External climate models

Rainfall model 625—Rainfall Model requires inputs from location, Microclimate model, and NOAA climate forecast

Soil nutrient model 630—Soil Nutrient Model f(t)—Requires Location, LiDAR surface contour, Crop Maturity Model, Water transport model

Pest infestation 640—Pest infestation scan requires inputs from Location, Visual Imagery, Spectroscopy

Chemical application 645—Pest chemical application requires inputs from Location, Microclimates, External climate models.

Farm practices optimization 650—Farm Practices Optimization—Requires input from Soil Type and Soil Moisture and LiDAR, and ground surface condition (fanner input).

Crop health 655—Crop Health/Stress—Requires input from Location, Spectroscopy, Soil Moisture, Soil Nutrient data base.

Crop yield simulator 660—Crop Yield Simulator—requires input from all inputs described in this document.

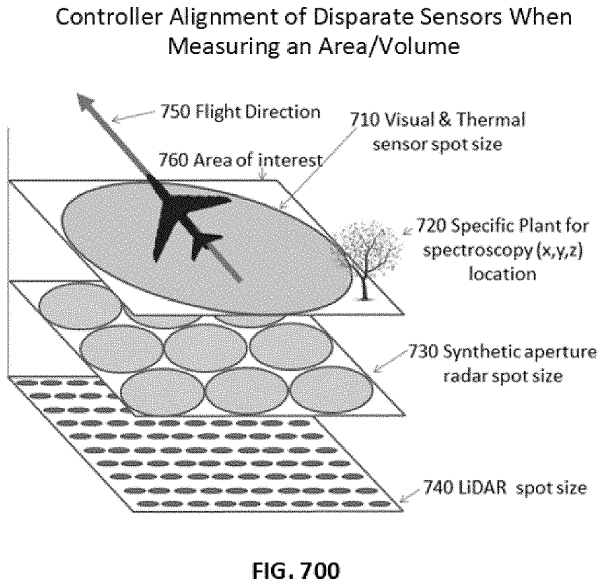

FIG. 700 shows controller alignment of disparate sensors when measuring a volume. FIG. 700 shows the vehicle direction and location 750. On board calculation of location is performed by the sensor computer FIG. 525/565. The computer calculates when to enable each of the sensors that it controls. The computer first takes into account the area of interest 760 then, knowing each sensor focal length and spot size the computer plans a scan for each of the multiple on board sensors and as the vehicle comes into position executes the plan. An example of this is to look at three spot sizes 740 shown in FIG. 700. The largest spot size are the visual and thermal sensor spot sizes 310. This requires only one picture in the area of interest 760. The synthetic aperture radar spot size 730 at this altitude requires 9 measurements as shown in FIG. 700 and therefore the controller schedules nine per area of interest 760. The last sensor is the LiDAR 740, which is a scan even though; the LiDAR requires start stop timing.

Focal point calculations are required for the radar as a function of aircraft height and therefore the controller has to adjust number of spots proportionally to distance from target. For instance, if the distance is 500 feet in altitude the radar measures 49 spots, if the altitude is 1000 feet the radar must be set up to measure 9 spots.

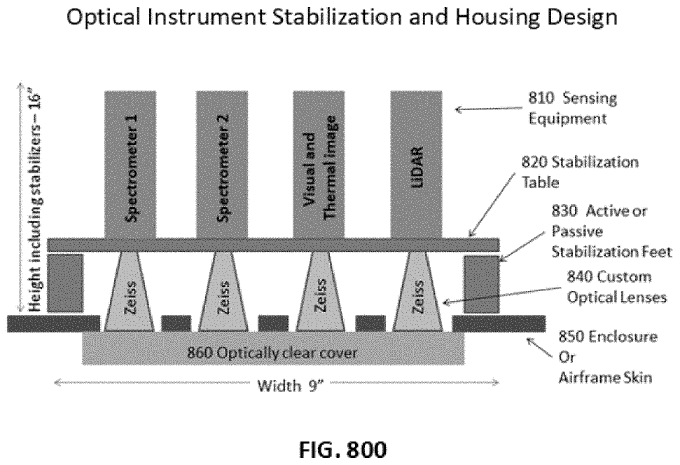

FIG. 800 illustrates an optical instrument stabilization and housing design. Optical instruments used in conjunction with other measurements must be stabilized from vibration to minimize/eliminate blur caused by vibration of vehicle in motion. FIG. 800 shows a simplified version of the design however this design extends to mounting to all devices individually or separate onto a stabilization table 820 which uses multiple passive or active stabilization feet 230.

Imaging equipment is typically also sensitive to dust so the enclosure introduced in FIG. 800 includes the use of an optically transparent cover which is attached to the enclosure FIG. 250 such that no air or water or dust might leak in.

Custom lenses 840 are specially adapted to the imager components 210. These lenses allow for adjustment of focal points such that each piece of sensing equipment might focus at the proper range and exhibit a designed for spot size.

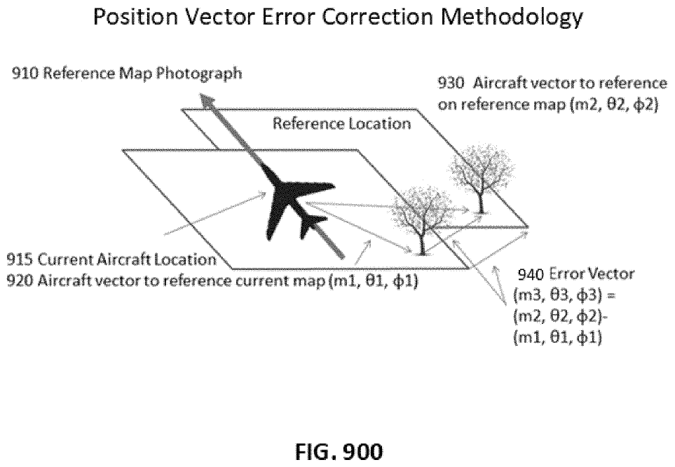

FIG. 900 shows the position vector error correction methodology utilizing two sensors referenced herein. This methodology requires a reference location image 910 stored prior to scanning and a photograph of the current aircraft location to be taken when the vehicle is located within range of the GPS target location 920. The system then correlates one spot or one surface on the reference map to one spot or one surface on the current measured picture and generates two vectors Vr and Vp in three-dimensional space. The error vector Verr is the difference between the two.

Once the difference vector 940 is found, all location measurements are adjusted by subtracting the error vector 940 from the GPS location vector.

While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

What is claim is:

1. A data collection system for collecting and analyzing farm terrain data, comprising:
location data for a plurality of measured spots that aggregate to represent farm terrain;
ground penetrating RADAR data for the measured spots of farm terrain;
microclimate data for the measured spots of farm terrain;
soil moisture and soil type data for the measured spots of farm terrain;
environmental data for the farm terrain;
crop type data for the farm terrain; and
wherein the data collection tool provides the location data, radar data, microclimate data, soil moisture data, soil type data and environmental data to simulate growth and maturity of the crop type for the farm terrain.

2. The data collection system according to claim 1, further comprising LiDAR data, which is further provided to the farm crop simulation tool.

3. The data collection system according to claim 1, further comprising data from a plurality of RADARs, which is further provided to the farm crop simulation tool.

4. The data collection system according to claim 1, wherein the RADAR deploys multiple antennas in order to electronically steer the measurement location.

5. The data collection system according to claim 1, wherein the RADAR is a synthetic aperture RADAR which is controlled by the controller to electronically steer the measurement location

6. A vehicle for collecting data for a plurality of measured volumes that aggregate to represent farm terrain, comprising:
instruments for collecting information regarding each measured volume, comprising:
a GPS receiver for collecting location data;
ground penetrating RADAR for collecting soil type data and soil moisture data;
optical camera for collecting visible information;
thermal imager; and
spectrographic imager;
a controller connected to the instruments; and
wherein the controller, according the each instrument's latency and resolution, determines when each instrument will be triggered to collect information regarding each measured volume.

7. The vehicle according to claim 6, wherein the instruments further comprise LiDAR, and the controller further triggers the LiDAR to collect data according to its latency and resolution.

8. The vehicle according to claim 6, wherein the instruments further comprise multiple RADAR antennas for signal processing to create a larger aperture, and the controller further triggers each RADAR to collect data according to its latency and resolution.

9. The vehicle according to claim 6, wherein the instruments further comprise multiple RADAR antennas for signal processing to synthetic aperture, and the controller further triggers each RADAR to collect data according to its latency and resolution.

10. The vehicle according to claim 6, wherein the vehicle is a ground vehicle.

11. The vehicle according to claim 6, wherein the vehicle is an airplane, drone or balloon.

12. The vehicle according to claim 11, further comprising stabilization system for optical instruments, further comprising:
passive stabilization feet connected to the vehicle;
a stabilization table attached to the stabilization feet; and
a plurality of optical instruments mounted on the stabilization table.

13. The vehicle according to claim 12, wherein the plurality of optical instruments are selected from the group consisting of: optical camera, spectrometers, LiDAR and thermal sensor.

14. A method of correlating disparate sensors for a data collection system that is collecting farm terrain data for an area of interest, comprising:
connecting a plurality of sensing instruments connected to a common controller, the instruments further comprising two or more of:
ground penetrating RADAR;
optical camera;
thermal imager; and
a spectrographic imager;
determining, for each instrument, the number of collection spots needed to cover the area of interest;
triggering, using the controller, each instrument according to its latency and spot size;
collecting data from each instrument, and time stamping the collected data;
comparing the data collected from the optical camera to a stored reference image to generate an error vector between the actual image data and the reference image data; and
adjusting location data for the collected data using the error vector.

15. The method according to claim 14, further including stamping the collected data with GPS location information.

16. The method according to claim 14, further including adjusting the stamped GPS location data by the error vector.

17. The method according to claim 14, further comprising changing the number of measurement spots for the RADAR according to the RADAR's height above the area of interest.

18. The method according to claim 14, wherein the instruments further comprise LiDAR, and the controller further triggers the LiDAR to collect data according to its latency and resolution.

19. The method according to claim 14, wherein the instruments further comprise multiple RADAR antennas for signal processing to create a larger aperture, and the controller further triggers each RADAR to collect data according to its latency and resolution.

20. The method according to claim 14, wherein the instruments further comprise multiple RADAR antennas for signal processing to synthetic aperture, and the controller further triggers each RADAR to collect data according to its latency and resolution.
